(19) United States
(12) Patent Application Publication
(10) Pub. No.: US 2021/0231599 A1
(43) Pub. Date: Jul. 29, 2021

(54) Biosensor for the Detection of an Analyte

(71) Applicant: LeNano Diagnostics Inc., Mississauga (CA)

(72) Inventor: Xiaowu Tang, Waterloo (CA)

(21) Appl. No.: 17/052,739

(22) PCT Filed: May 3, 2019
(86) PCT No.: PCT/CA2019/050595

§ 371 Date: Nov. 3, 2020

Related U.S. Application Data
(60) Provisional application No. 62/667,143, filed on May 4, 2018.

(51) Int. Cl. G01N 27/327; G01N 33/543; G01N 27/02

(52) U.S. Cl. CPC G01N 27/3276; G01N 27/02; G01N 27/3278; G01N 33/5438

(57) ABSTRACT

A biosensor useful for the detection of a target analyte in a fluid biological sample is provided. The biosensor comprises a first sensing layer comprising at least a pair of metal electrodes on an inert substrate, wherein the electrodes are linked by an electrically conductive bridge having immobilized on the surface thereof analyte-reactive compounds, wherein the electrically conductive electrode bridge comprises a material that responds to binding of target analyte to the analyte-reactive compound with an electrochemical or electrical change; and, optionally, a second layer comprising one or more sample chambers for placement on the first sensing layer, wherein the sample chambers are adapted to receive a fluid sample and direct the sample to the bridge. The biosensor may be incorporated in a point of care device additionally comprising a detection device that forms an electrical circuit with the biosensor, provides an electrical signal over a range of frequencies and is adapted to detect an electrochemical or electrical change at the electrode bridge in the presence of target analyte using electrochemical impedance spectroscopy on application of the electrical signal.

### FIELD OF THE INVENTION

The present invention generally relates to sensing systems and methods, and in particular, to a novel biosensor, a device incorporating the biosensor and methods for use to detect an analyte in a sample.

### BACKGROUND OF THE INVENTION

Heart failure (HF) occurs when the heart can no longer pump blood adequately to meet the needs of the body. It is a global pandemic that affects at least 26 million people worldwide with increasing prevalence. The cost of HF on the economy is significant and will inflate dramatically with the aging population. Despite the significant advances in therapies and prevention, patients still suffer from poor quality of life, and high mortality and morbidity rates. Accurate and early diagnosis is of vital importance for improving outcomes of HF patients. However, HF diagnosis can be challenging because HF symptoms such as shortness of breath (dyspnea) or fatigue are subjective and non-specific. For instance, dyspnea may be found in individuals who are obese or have chronic obstructive pulmonary disease but do not suffer from HF.

Over the past two decades, numerous studies have shown that brain natriuretic peptide (BNP) is an effective diagnostic and prognostic biomarker for HF. BNP is a neurohormone secreted by the heart's ventricles in response to decreased blood pressure or increased systemic vascular resistance. There is a direct correlation between elevated concentrations of BNP in blood (>400 pg/mL) and the severity of HF symptoms. However, a gray zone exists (100-400 pg/mL) where elevated plasma BNP levels cannot be attributed to HF but can still lead to early prognosis of decompensation. Point-of-care (POC) monitoring of BNP levels is crucial since HF patients have fluctuating plasma BNP concentrations everyday. It will enable efficient and timely management of HF, as well as prevent potential hospitalization.

Currently, Food and Drug Administration (FDA)-approved immunoassays for plasma BNP detection are based on optical methods which require particular instrumentation and specially trained personnel. These existing methods are sandwich immunoassays which require both capture and labelled detection anti-BNP antibodies that can be expensive.

A promising alternative is to use electrical measurement-based sensors. Electrochemical impedance spectroscopy (EIS) is a versatile and sensitive tool widely used in different fields, particularly in sensors. It can monitor the surface bio-recognition events on a modified electrode surface in terms of charge transfer, conductance and electrical double layer capacitance change. For example, Periyakaruppan and colleagues (Anal. Chem. 2013. 85(8):3858-63) produced a label-free, carbon nanofiber (CNF)-based immune-sensor that uses EIS to detect cardiac troponin I (cTnI). The detection limit of this immune-sensor was 25 times lower than that achieved by conventional methods, enabling detection of cTnI at a concentration of ˜0.2 ng/mL. Gupta and coworkers (Biosens. Bioelectron. 2014, 59, 112-119) used combined EIS and carbon nanoarray to quantify C-reactive protein (CRP), another cardiac biomarker. The device had a detection limit of ˜11 ng/mL, which was considered clinically relevant.

Low-cost, easy-to-use, sensitive and/or accurate sensing devices would be desirable, but have yet to be developed.

### SUMMARY OF THE INVENTION

A novel biosensor for detection of an analyte in a patient sample has now been developed and may be incorporated in a portable sensing device for the rapid detection of an analyte in a patient sample.

Thus, in one aspect of the present invention, a biosensor useful for the detection of a target analyte in a biological sample is provided comprising:
i) a first sensing layer comprising at least a pair of metal electrodes deposited on an inert substrate, wherein the electrodes are linked by an electrically conductive bridge having immobilized on the surface thereof analyte-reactive compounds, wherein the bridge comprises a material that responds to binding of target analyte to the analyte-reactive compound with an electrochemical or electrical change; and, optionally,
ii) a second layer comprising one or more sample chambers for placement on the first layer, wherein the sample chambers are adapted to receive a fluid sample and direct the sample to the bridge.

In another aspect of the invention, a point of care sensing device is provided comprising:
i) a first sensing layer comprising at least a pair of metal electrodes deposited on an inert substrate, wherein the electrodes are linked by an electrically conductive bridge having immobilized on the surface thereof analyte-reactive compounds, wherein the bridge comprises a material that responds to target analyte binding with an electrochemical or electrical change; and, optionally, a second layer comprising one or more sample chambers for placement on the first layer, wherein the sample chambers are adapted to receive a fluid sample and direct the sample to the bridge; and
ii) a detection device that forms an electrical circuit with the biosensor and is adapted to detect impedance over a range of frequencies that result from the electrochemical or electrical change at the bridge for analysis by Electrochemical Impedance Spectroscopy (EIS).

In another aspect of the invention, a method of detecting a target analyte in a patient sample is provided comprising:
i) applying the sample to a biosensor as defined above;
ii) collecting electrochemical signal data from the biosensor in the presence of the sample over a range of frequencies; and
iii) analyzing conductivity, resistance and capacitance based on the electrochemical signal data using Electrochemical Impedance Spectroscopy (EIS) and determining the presence of target analyte in the sample when the impedance or overall resistance is different in the absence and presence of the sample.

These and other aspects of the invention will become apparent in the detailed description that follows by reference to the following figures.

### BRIEF DESCRIPTION OF THE FIGURES

FIG. 1 illustrates: (a) AFM image of Chemical Vapor Deposition (CVD) grown CNT-TF; (b) SEM image of CVD grown CNT-TF; and (c) Raman spectrum of the CVD grown CNT-TF;

FIG. 2 is a flow chart illustrating CNT-TF (carbon nanotube thin film) sensor fabrication;

FIG. 3 illustrates: (a) equivalent circuit of the CNT-TF sensor; (b) a fitted Nyquist plot in MATLAB arising from the equivalent circuit; and (c) fitted Nyquist diagrams of CNT-TF sensor at each step during the sensor preparation and BNP detection;

FIG. 4 illustrates: (a) a standard addition plot for an immunofluorescence assay; and (b) standard addition plot for the CNT-TF sensor;

FIG. 5 graphically illustrates BNP values as determined using CNT-TF sensors vs ELISA for standard BNP samples in plasma;

FIG. 6 graphically illustrates BNP values as determined using CNT-TF sensors vs Alere Triage® for patient plasma samples;

FIG. 7 graphically illustrates BNP values determined using CNT-TF sensors vs theoretical BNP values for standard NT-proBNP samples in plasma;

FIG. 8 illustrates A) layers of a sample chamber embodiment; and B) an integrated sample chamber embodiment:

FIG. 9 illustrates an embodiment of a biosensor; and

FIG. 10 illustrates via a flow chart the connection of a biosensor and data reader in accordance with an embodiment.

### DETAILED DESCRIPTION OF THE INVENTION

A biosensor useful for the detection of a target analyte in a biological sample is provided. The biosensor comprises: i) a first sensing layer comprising at least a pair of metal electrodes deposited on an inert substrate, wherein the electrodes are linked by an electrically conductive bridge having immobilized on the surface thereof analyte-reactive compounds, wherein the bridge comprises a material that responds to binding of target analyte to the analyte-reactive compound with an electrochemical or electrical change; and, optionally, ii) a second layer comprising one or more sample chambers for placement on the first layer, wherein the sample chambers are adapted to receive a fluid sample and direct the sample to the bridge.

The first sensing layer comprises a substrate which is an inert, supportive material, e.g. suitable to support electrodes attached to or integrated within the material. Examples of suitable materials include polymeric materials such as thermoplastic polymers, including but not limited to, polyesters such as polyethylene terephthalate (PET), acrylic, acrylonitrile butadiene styrene, polyamides, polylactic acid, polycarbonate, polyethylene, polystyrene, polypropylene, polyvinyl chloride, polyether sulfone, polyoxymethylene, polyetherether ketone, polyetherimide, polyphenylene oxide or sulfide, polybenzimidazole, polydimethylsiloxane (PDMS), expoxy, polyethylene glycol (PEG), hydroxypropyl cellulose (HPC), poly(N-isopropylacrylamide), silica (silicon dioxide), and the like.

The first sensing layer of the biosensor also comprises at least a pair of electrodes, either applied to substrate, or incorporated within the substrate. For example, the electrodes may be screen-printed, physically or chemically evaporated onto the substrate, or the substrate may be etched with a suitable pattern to incorporate the electrode within the substrate. The electrodes may be made out of any suitable conductive metal such as carbon, gold, platinum, silver, or a combination thereof. In one embodiment, the width of the electrodes may be about 250 μm, preferably greater than 500 μm, such as 750 μm. The gap between electrodes is at least about 500 μm, and preferably in the range of about 1-1.5 mm.

The biosensor may comprise a pair of electrodes, i.e. a working electrode and a counter or auxiliary electrode, or 2 or more electrode pairs. The electrodes may assume various functional arrangements on the first substrate layer as will be appreciated by one of skill in the art. For example, the electrodes may be arranged in pairs, either alternating pairs or a series of working electrodes adjacent to a series of counter electrodes. Alternatively, a series of working electrodes may be arranged in an array with a single common counter electrode. In this regard, the biosensor may include multiple arrays with 2 or more common electrodes.

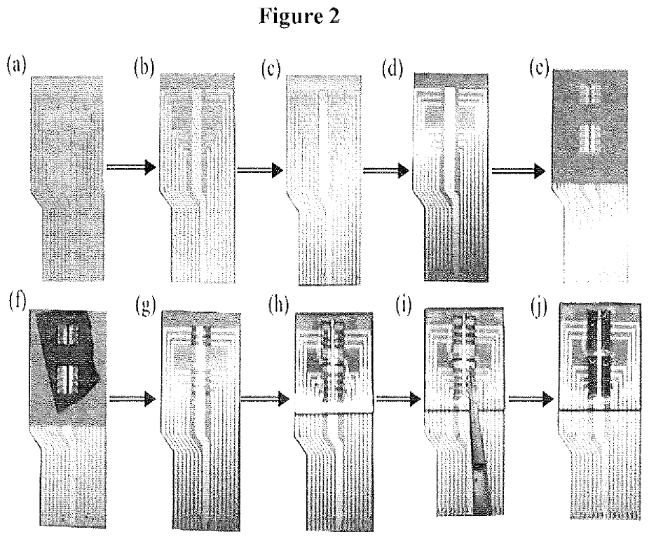

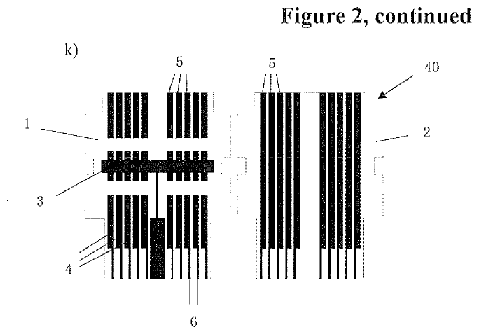

The electrode pairs may be situated on the same side of the biosensor substrate as illustrated in the biosensor of FIG. 2(j), or may be situated on both sides of the substrate as shown in FIG. 2(k). By reference to FIG. 2(k), the first side (1) of the substrate comprises a common counter electrode (3) to which multiple working electrodes (4) and (5) are connected via electrically conductive bridges. Both sets of electrodes connect to the power source of the data reader or detection device via electrical connections (6) on the first side (1) of the substrate for electrodes (4) and on the second side (2) of the substrate for electrodes (5). Electrodes (5) from the first side of the substrate (1) are similarly connected to the second side (2) of the substrate by a suitable electrical connection, for example, a FFC/FPC connector.

Each working and counter electrode are connected by an electrically conductive bridge. The electrically conductive bridge may be made of a carbon-based material, such as graphene, reduced graphene oxide or a carbon nanotube network, as well as silica (e.g. thin film or nanowires) and molybdenum disulfide (MoS₂). In one embodiment, the electrodes are connected by a carbon nanotube network comprising carbon nanotubes (e.g. in the form of cylindrical pipes, 1-10 nm in diameter and 2-10 microns in length). Nano-sized channels or pores, e.g. 1-5 nm in diameter, form between the nanotubes through which sample can pass to permit reaction of the target analyte with immobilized analyte-reactive species immobilized on the electrically conductive bridge, e.g. a nanotube network. The nanotubes may be randomly oriented, semi-aligned or parallel. Carbon nanotubes (CNTs) advantageously provide a high surface-to-volume ratio and low density of electronic states, which contribute to high sensitivity for sensor applications. CNT-based biosensors have demonstrated an ultralow limit of detection (LOD) in the range of femtomolar (fM) to attomolar (aM) for a wide variety of analytes.

Immobilized on the bridge connecting the electrodes are analyte-reactive compounds that result in an electrochemical or electrical signal change (impedance change) in response to target analyte binding thereto. The target analyte is not particularly restricted. The target analyte may, for example, be a biomolecule such as nucleic acid, protein/peptide, carbon hydrates, lipids, or a microorganism such as bacteria, fungi or virus. Biological samples that may be analyzed for the presence of a target analyte using the present biosensor include fluid samples such as water samples, aqueous extracts from various sample types such as foods, plants, soil, etc., and bodily fluid samples such as blood, plasma, serum, saliva, urine, tears, breast milk, cerebrospinal fluid, amniotic fluid and ascitic fluid.

The analyte-reactive compound or species is a compound that will selectively react with the target analyte to effect an electrochemical or electrical signal change (change in impedance). For nucleic acid analytes, the analyte-reactive compound may be an oligonucleotide (DNA or RNA) fragment that is complimentary to a portion of the analyte. For protein/peptide analytes, the analyte-reactive compound may be a monoclonal or polyclonal antibody, a peptide ligand or receptor, an enzyme or substrate or other entity that exhibits binding specificity to the protein analyte. For a microorganism analyte, the analyte-reactive compound may be a ligand that binds a surface receptor on the microorganism, a receptor that binds a surface localized ligand on the microorganism or an antibody. The analyte-reactive compound may be immobilized on the electrically conductive bridge employing various methods including covalent linkage, electrostatic attraction, ionic bonding, hydrogen bonding, or van der Waals' forces.

In one embodiment, the biosensor is utilized to identify disease biomarkers in blood samples. Examples of biomarkers that may be detected include biomarkers of heart failure such as brain natriuretic peptide (BNP) and NT-proBNP, biomarkers for cardiac disease such as CK-MB (creatine kinase-muscle/brain) and Troponin I, as well as DNA tumor biomarkers. In another embodiment, the biosensor is utilized to identify virus and bacteria in fluid samples such as water or body fluid samples. Examples of bacteria that may be detected include, but are not limited to, Escherichia coli. Listeria sp., Salmonella sp., Staphylococcus sp. and Pneumococcus sp., and examples of viruses that may be detected include influenza viruses and rotavirus.

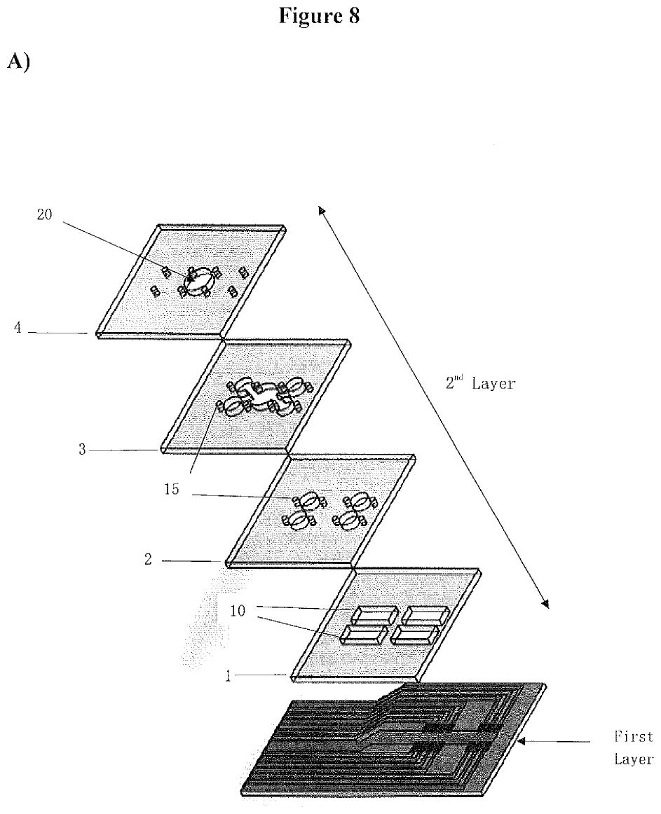

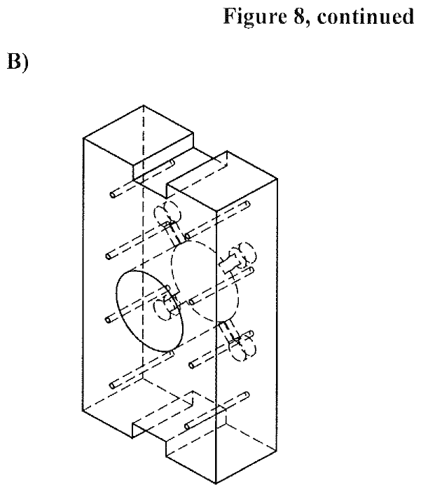

The biosensor comprises a second layer or element that incorporates one or more sample chambers adapted to receive the biological sample and guide the sample to the first sensing layer for exposure to the analyte-reactive compound immobilized on the electrically conductive bridge(s) connecting the electrodes. The sample chamber layer may be an integrated layer, or may itself comprise two or more layers. For example, as shown in FIG. 8A, the sample chamber layer may comprise a single layer (1) preferably made of a material that seals to the first layer such as an elastic material, e.g. rubber or silicone to prevent sample leakage of the sample on application. The sample chamber layer comprises 1 or more chambers (10) formed therein to receive the sample to be analyzed. The sample chamber layer may include 1 or more additional layers (2-4) to adapt the sample chamber, for example, to provide a single sample inlet (20) that divides into multiple chambers (10). The additional layers may be made of a non-deformable material, e.g. a thermoplastic polymeric material similar to the material of the first layer as described above, including acrylic (poly(methyl methacrylate) (PMMA)) and the like. As shown, the chambers in each layer are gradually altered to become a single inlet (20) in layer 4. Each layer may include air venting apertures (15). As will be appreciated by one of skill in the art, the sample chamber layer may also be prepared as an integrated microfluidic layer using established techniques to provide a single inlet that branches into 2 or more chambers such that sample applied to the inlet will flow into separate chambers for contact with separate electrodes or electrode arrays as shown in FIG. 8B.

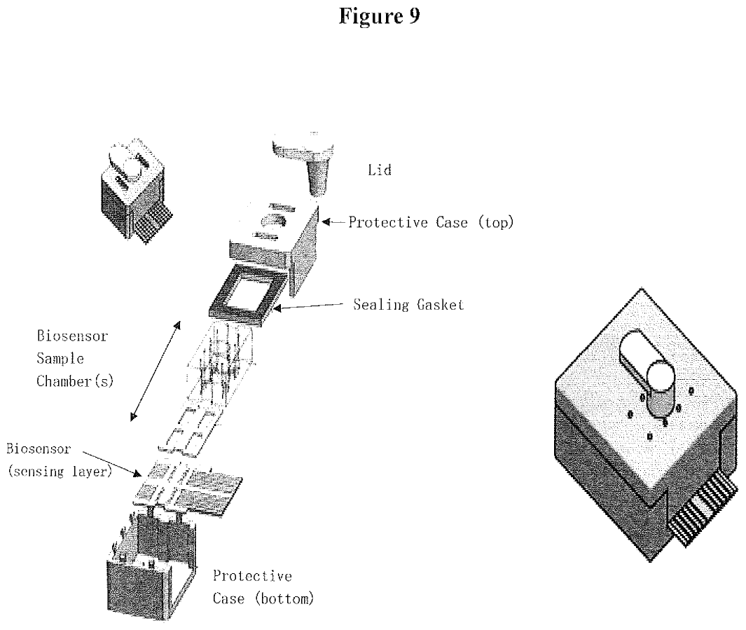

An embodiment of the biosensor is shown in FIG. 9. As shown, the first sensing layer and the sample chamber layer of the biosensor may be encapsulated within a protective case having a bottom and top. An aperture is formed in the top of the protective case which lines up with the sample inlet in the sample chamber layer. A lid to cap the aperture may be provided to prevent leakage of sample from the biosensor, or to prevent contaminants from getting into the biosensor. The lid may also provide pressure to push the sample into the sample chambers. The protective case is made of a hard polymeric material such as the thermoplastic materials of the first sensing layer. The protective case not only provides protection to the sensing layer, but also functions to compress the components of the biosensor together to ensure operability of the biosensor. In this regard, the biosensor may additionally comprise a sealing gasket between the sample chamber layer and the top of the protective case to enhance the seal/compression of the biosensor components.

The sample chambers incorporated within the second layer may have various configurations depending on the layout of the first sensing layer and its electrodes, and may include any number of chambers (e.g. from 1-10 or more) of various sizes (e.g. 1-100 μL or more) and shapes (e.g. rectangular, square, circular, oval, irregular). In a biosensor comprising a single chamber layer, the sample chambers formed therein may each be sized to accept a sample of about 10-20 μl. In a biosensor comprising a single sample inlet that branches into multiple sample chambers, the sample inlet will generally accommodate larger sample sizes (e.g. 50-100 or more) that will then be divided into the multiple chambers.

The present biosensor may assume various arrangements and configurations. For example, while the biosensor may comprise a single sample chamber that feeds sample to flow through the electrode bridge of a single electrode pair, the biosensor may also comprise a sample chamber that feeds sample to multiple electrode pairs, e.g. arranged in groups, for example, 2-5 electrode pairs, or may comprise multiple sample chambers, each feeding sample to 1 or multiple biosensor electrode pairs. The response of biosensors comprising such electrode pair groupings may be analyzed and together provide improved accuracy.

A multiplexing capability can also be implemented using the present biosensor, permitting detection of multiple biomarkers simultaneously. For example, multiple biosensor electrode pairs or arrays may be incorporated in the sensing layer of a biosensor, and the electrically-conductive bridges of each pair or array may be labelled with different analyte-reactive compounds to target various target analytes in a single sample. The sample chamber layer/element may be configured, as described, to divide a single sample into separate chambers, wherein each chamber feeds a portion of the sample to a biosensor electrode pair or array having a different analyte-reactive compound immobilized on their electrode bridges. In this way, the biosensor permits analysis of various analytes in a single sample.

The biosensor is adapted for linkage to, or is connectable with, an electronic data reading or detection device that completes an electrical circuit with the biosensor and is adapted to detect impedance over a range of frequencies that result from the electrochemical or electrical change at the electrically-conductive electrode bridge for analysis by Electrochemical Impedance Spectroscopy (EIS). The detection device functions to: 1) provide excitation signals to the biosensor in order to measure electrochemical modulation at the electrode bridge surface, and 2) receives electrical signal from the biosensor for analysis by electrochemical impedance spectroscopy (EIS) in order to provide an output signal that indicates the presence or absence of analyte in a sample being tested, e.g. based on the detection of a change in the electrical signal, which modulates the conductivity, resistance, and/or capacitance of the electrode bridge. Thus, the detection device is adapted to provide excitation signals over a range of frequencies, receive electrical signal from the electrode(s) and conduct impedance measurement thereof to generate an EI spectra from which capacitance and resistance is calculated and which permits analyte determination based on correlation with the signal. As one of skill in the art will appreciate, analysis of the EI spectra is based on fitting with an equivalent circuit which can readily be done using available software, such as MATLAB, NOVA, Python, ZSimpwin, etc.

Thus, the biosensor is adapted to electrically connect to the data reader via electrical connection means such that the electrodes form a circuit with a power source (e.g. AC source) in the data reader/detection device. While the biosensor may be electrically connected to the reader in any suitable fashion, generally the connection will utilize a flat flexible cable (FFC) for connection to a flexible printed circuit (FPC). The FFC generally consists of a flat and flexible plastic film base, with multiple metallic conductors bonded to its surface. Each end of the cable may be reinforced with a stiffener to facilitate the insertion and connection of the biosensor to the reader and provide strain relief. The parameters of the FFC, such as the pitch, number of pins, material, width and depth will depend on the design of biosensor. Some generally used parameters include a pitch or 0.5 mm or 1 mm and 6 to 30 pins (e.g. metallic electrical conductors) made of copper, lead or zinc.

Exposure and interaction of the analyte-reactive compounds immobilized on the biosensor electrode bridge(s) with a target analyte results in a change of the interfacial properties of the electrode that results in an electrochemical or electrical signal change that is proportional to the concentration of analyte in the sample, and which is measurable by the detection device. The interaction between the analyte-reactive compound and the analyte modulates the conductivity, resistance, and/or capacitance of the biosensor, for example by a change in pH, electrical charge, or by deformation of the reactive species, which is then determined using electrochemical impedance spectroscopy (EIS). EIS measures the resistance and capacitance properties of the electrode via application of the AC excitation signal, e.g. of less than 20-50 mV, such as 2-10 mV, over a range of frequencies (e.g. from 80-60,000 Hz, such as 200 Hz to 10,000 Hz), to generate an impedance spectrum from which the capacitance and resistance of the system can then be calculated. The change in conductivity, resistance, and/or capacitance of the electrode bridge is then extracted from the EIS spectra, which advantageously isolates the electrode bridge resistance change in the midst of other changes.

Thus, in use, a quantity of sample, e.g. a blood sample, to be analyzed for the presence of a target analyte, is administered to the biosensor at the sample chamber(s), or at the sample inlet. The sample will contact the electrically conductive electrode bridge having analyte-reactive compound immobilized thereon. In the absence of target analyte in the sample, there will be no change in the electrical or electrochemical properties at the electrode bridge, and no change in impedance detected/calculated by the reader/detection device to which the biosenser is connected. On the other hand, in the presence of target analyte in the sample, the analyte will interact with the analyte-reactive compound on the electrode bridge causing a change in the electrical or electrochemical properties thereof, which will be reflected in the impedance detected by the reader/detection device and which will correlate with the level of analyte in the sample.

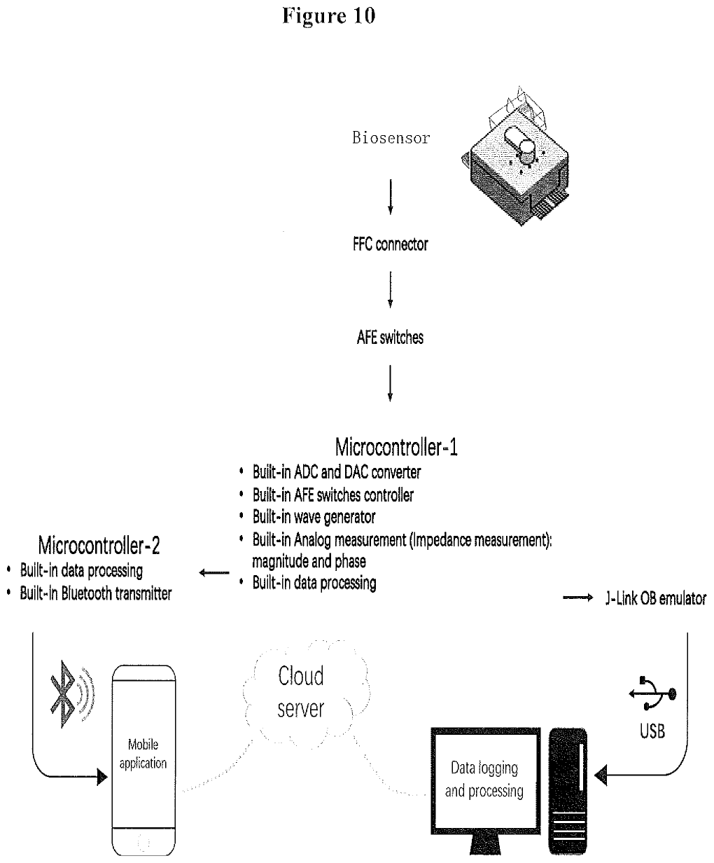

As will be appreciated by one of skill in the art, the present biosensor with data reader may be incorporated into a point of care testing (POCT) device, which may include a microprocessor (e.g. digital signal processor) or digital acquisition board to digitize the signal from the reader, and a display unit, such as a monitor, which is in communication with or connected to the microprocessor, and functions to display the signal in a suitable format as exemplified in the flow chart of FIG. 10.

Alternatively, as will be understood by a person of skill in the art, the data reader may be separate from the display unit, and in communication with an external display unit for presenting the output of the signal generating reader thereon. For convenience, the monitor may be portable, and battery operated. According to another embodiment, the data reader may further comprise an algorithm processing module for receiving information via a user interface, for example. The algorithm processing module is operable to translate the signal received from the biosensor into a desired output.

The reader can be implemented on one or more respective computing device(s) which can include a network connection interface, such as a network interface card or a modem, coupled via connection to a device infrastructure. The connection interface is connectable during operation of the device(s) to a network (e.g. an intranet and/or an extranet such as the Internet) which enables the device(s) to communicate with each other as appropriate. The network can, for example, support the communication of the output signal provided by the biosensor to the reader.

The device(s) may also have a user interface coupled to the device infrastructure to interact with a user. The user interface can include one or more user input devices such as, but not limited to, a QWERTY keyboard, a keypad, a trackwheel, a stylus, a mouse, a microphone and a user output device such as an LCD screen display and/or a speaker. If the screen is touch sensitive, then the display can also be used as the user input device as controlled by the device infrastructure.

Operation of the device(s) is facilitated by the device infrastructure. The device infrastructure includes one or more computer processors (e.g. a Digital Signal Processor) and can include an associated memory (e.g. a random access memory). The computer processor facilitates performance of the computing device configured for the intended task through operation of the network interface, the user interface and other application programs/hardware of the computing device by executing task-related instructions. These task-related instructions may be provided by an operating system and/or software applications located in the memory, and/or by operability that is configured into the electronic/digital circuitry of the processor(s) designed to perform the specific task(s). Further, it is recognized that the device infrastructure may include a computer readable storage medium coupled to the processor for providing instructions to the processor. The computer readable medium can include hardware and/or software such as, by way of example only, magnetic disks, magnetic tape, optically readable medium such as CD/DVD ROMS, and memory cards. In each case, the computer readable medium may take the form of a small disk, floppy diskette, cassette, hard disk drive, solid-state memory card or RAM provided in the memory module. It should be noted that the above listed examples of computer readable media may be used either alone or in combination. The device memory and/or computer readable medium may be used to store, for example, the output of the biosensor/reader for use in processing of the signal.

Further, it is recognized that the computing device(s) may include executable applications comprising code or machine readable instructions for implementing predetermined functions/operations including those of an operating system. The processor as used herein is a configured device and/or set of machine-readable instructions for performing operations as described by example above. As used herein, the processor may comprise any one or combination of, hardware, firmware, and/or software. The processor acts upon information by manipulating, analyzing, modifying, converting or transmitting information for use by an executable procedure or an information device, and/or by routing the information with respect to an output device. The processor may use or comprise the capabilities of a controller or microprocessor, for example. Accordingly, the functionality of the reader may be implemented in hardware, software or a combination of both. It will be understood that the computing device(s) may be, for example, a personal computer, personal digital assistant, mobile phone, and/or content player. Further, it is recognized that each server computing device, although depicted as a single computer system, may be implemented as a network of computer processors, as desired.

The present invention represents the first time that an electrically conductive biosensor, comprising an electrically conductive bridge connecting working and counter electrodes, is utilized with EIS to provide an accurate reading of the electrochemical or electrical change on the biosensor.

The present biosensor exhibits a number of advantages. First, it has been determined by comparison to other analyte detecting techniques (such as optical immunoassay) that the present biosensor generates both highly accurate and sensitive results. It also enables detection of analyte in complex samples such as blood/plasma without requiring sample preparation (e.g. purification) prior to analysis to avoid background noise. In addition, the biosensor provides for analyte detection without the need for any additional labeling or amplification to enable detection. Further, signal detection arises upon analyte binding to the analyte-reactive compound without the need for subsequent washes to remove unbound species to eliminate false-positive signals.

Embodiments of the invention are described by reference to the following specific example which is not to be construed as limiting.

### EXAMPLE

Materials and Chemicals

Iron(III) acetylacetonate (51003), molybdenum(II) acetate (2320761), cobalt(II) acetate (3999731), fumed silica (7 nm in diameter; S5130), BNP (B5900), bovine serum albumin (BSA; A2153), Trizma base (T6066), hydrochloric acid (258148) and StabilCoat® Immunoassay Stabilizer (S0950) were all purchased from Sigma Aldrich. PBS buffer (OX) was purchased from VWR LLC. Hydrofluoric acid (HX0621) was obtained from EMD. Silicon wafer (P/Boron dopant, 20000 angstroms thermal oxide) was obtained from Silicon Valley Microelectronics. Compressed hydrogen and argon were obtained from Praxair. Polyethylene terephthalate (PET; Melinex 329/1000, 10 mil thickness) was purchased from Tekra. Removable vinyl sticker (3 mil thickness) was purchased from Digital Graphic Inc./Sign Supply Canada. Optically clear adhesive (Type 8213, 3 mil thickness) was obtained from 3M. Poly (methyl methacrylate) sheet (PMMA; 4.75 mm thickness) was obtained from McMaster-Carr. Kimwipes were purchased from Kimtech. Antibody 50E1 was obtained from Abeam, while 24E11 and 24C5-biotin were obtained from Novus Biologicals. BNP, NT-proBNP, and NT-proBNP free plasma (BNFP) were obtained from Hytest Ltd. Patient plasma was purchased from BioreclamationIVT. COOH-coated polystyrene plates were purchased from Greiner-Bioworld. 1-Ethyl-3-(3-dimethylaminopropyl) carbodiimide (EDC) was purchased from GBiosciences. N-hydroxysuccinimide (NHS) was purchased from Alfa Aesar. Eu-Streptavidin and DELFIA® Enhancement Solution was purchased from Perkin Elmer.

### CNT-TF Synthesis and Characterization

CNT-TFs were synthesized using a chemical vapor evaporation method similar to that described by Mandal et al. J. Nanosci. Nanotechnol. 2011, 11(4), 3265-3272. Briefly, a cocktail of metal catalysts including iron (III) acetylacetonate (3.0 mg), molybdenum(II) acetate (0.75 mg) and cobalt(II) acetate (4.6 mg) was sonicated with silica fume nanoparticles (50 mg) in ethanol (10 mL, anhydrous) for two hours. The mixture was then spin-coated at 2500 rpm for 20 s onto a 2×3 cm rectangular piece of SiO₂/Si wafer (pre-cleaned by piranha solution). The wafer was transferred into a 2″ CVD quartz tube furnace and the temperature was ramped up to 850° C. under 600 standard cubic centimeters per minute (sccm) argon and 18 sccm hydrogen. After the temperature stabilized at 850° C., the gas flow was switched to bubble through an ethanol-contained bubbler (0° C.) to introduce ethanol vapor for 20 min. Then the carrier gases were switched to bypass the ethanol bubbler and the CVD system was cooled down to room temperature.

CNT-TF samples were characterized using three different methods to analyze purity and topography: Raman spectroscopy, atomic force microscopy (AFM) and scanning electron microscopy (SEM). For Raman and AFM characterizations, the CNT-TF on SiO₂/Si substrate was immersed in 1% hydrofluoric acid for 1 min, followed by a water bath to transfer the floating CNT onto another piece of clean SiO₂/Si wafer. Raman spectra with a wavenumber range between 1000 cm⁻¹ to 1800 cm⁻¹ was acquired by a Horiba Jobin Yvon LabRAM HR 800 Raman spectrometer with a 532 nm excitation laser. AFM images were collected using a Nanoscope MultiMode™ AFM instrument (Veeco) under tapping mode with a silicon probe tip at a resonant frequency of 300 kHz. For SEM characterization, the CNT-TF was transferred onto gold-coated polyethylene terephthalate (PET) and mounted on an SEM sample stage. Images were taken in a Zeiss ULTRA Plus SEM with a 10 kV accelerating voltage under 100 k× magnification.

### CNT-TF Sensor Fabrication

A 30 cm² PET (polyethylene terephthalate) sheet was cleaned with ethanol and dried with Kimwipe, followed by sticking a removable vinyl film onto it. Then the vinyl layer was patterned using a vinyl cutter (Graphtec CE6000-40) to form a screen mask for electrode deposition. After Cr/Au (40 nm/200 nm) deposition with an e-beam evaporator (Intlvac Nanochrome Deposition System), the screen mask was peeled off to form patterned Cr/Au electrodes. An additional layer of patterned and removable vinyl sticker was taped onto the Au to create 14 negative windows for CNT-TF transfer. Subsequently, the CNT-TF grown on SiO₂/Si substrates were immersed in 1% hydrofluoric acid for 1 min, followed by transfer onto the patterned vinyl-covered PET strip. The strip was dried on a hot plate (1000° C.) for 5 min and the vinyl mask was removed to form 14 microsensors. Each microsensor consisted of a CNT-TF bridging a pair of gold electrodes.

### Top Cover Fabrication

A 30 cm² PMMA (poly(methyl methacrylate) sheet was cleaned with ethanol and dried with Kimwipe, followed by sticking a double-sided adhesive to one side of the PMMA sheet. Then the sheet was patterned using a laser cutter (Universal laser system) to create four microsensor rectangular chambers. The dimensions (2.5 mm/6 mm) of the four chambers were identical to each other to eliminate volume variance interference.

### Antibody Immobilization and Surface Blocking

Capture antibody (50E1) was immobilized onto the CNT-TF sensor through physical absorption. Briefly, 10 pg/ml 50E1 in 1×PBS was added into strip sample chambers and incubated at 4° C. overnight. The next day, the chambers were washed with 5% BSA in 1×PBS three times and subsequently blocked for 2 hours with the same 5% BSA solution at room temperature. After blocking, the chambers were washed with 1×PBS three times before BNP sample addition.

### Characterization of CNT-TF Sensor

The as-prepared CNT-TF sensor was characterized using EIS by a customized impedance analyzer with an AC potential from 80 Hz to 60000 Hz. The reader's output of impedance and phase were collected over a range of the frequencies and saved on a computer. The collected spectra were simulated in MATLAB using an equivalent circuit proposed in this study and then the fitted CNT-TF resistance value was recorded against the detection signal of the sensor. Subsequently, a step-by-step characterization was performed on the sensor by collecting the following EIS spectra: 1) before and after antibody immobilization, 2) after BSA blocking, and 3) after the addition of 1000 pg/ml BNP in BNFP at 0, 5, 10 and 20 min.

As one of skill in the art will, fitting equations as follows were used. The total impedance impression of the bridge structure was as follows:

$${Z}_{total} = Rcir + \frac{1}{\frac{1}{Rcnt} + \frac{1}{Rs + \frac{1}{Cdl * {( j * w )}^{⋀} n}}}$$

Based on this impression, the fitting equation group used in MATLAB were as follow:

y=Zr−real(Zₜₒₜₐₗ)+Zi−imag(Zₜₒₜₐₗ)+Z−(real(Zₜₒₜₐₗ)\hat{ }2+imag(Zₜₒₜₐₗ)\hat{ }2)\hat{ }(½)

wherein Z is the tested impedance result from the reader, Zr is the real part of the tested impedance, calculated using the phase value, and Zi is the imaginary part of the tested impedance, calculated using the phase value. When the value of y reaches the minimum, the fitting is complete.

### Optical BNP Immunoassay

Capture antibody (50E1) was covalently immobilized onto COOH-coated polystyrene plates through EDC-NHS chemistry. The capture antibody at a concentration of 66.9 nM was mixed with 20 mM EDC and 50 mM NHS for 2 hours at room temperature. After washing with PBS-T three times, the wells were blocked with 5% BSA overnight at 4° C. The next day, the wells were washed twice with PBS-T. BNP was diluted in StabilCoat® Immunoassay Stabilizer at 0, 5, 7.5 and 10 ng/mL. A 10 μL drop of each concentration was placed on a circular hydrophobic PMMA and dried for 2 hours in a vacuum desiccator. 100 μL of unknown BNP concentration (X pg/mL) sample containing 6.66 nM of 24C5-biotin antibody were added to each well. The dried BNP on the hydrophobic circular PMMA was then added to the wells to produce X, X+500, X+750, X+1000 pg/mL BNP samples and incubated for one hour at room temperature. After incubation, the hydrophobic circular PMMA were removed and wells were washed with 20 mM Tris-HCl buffer three times. The PMMA was further incubated with 5.56 nM Eu-Streptavidin diluted in 50 mM Tris-HCl for 30 min at room temperature. The wells were then washed with 50 mM Tris-HCl and incubated with DELFIA® enhancement solution for 1 hour at room temperature. Fluorescent signals were recorded using a Spectramax M2e on the time-resolved fluorescence setting with 340 nm excitation and 615 nm detection wavelengths. To determine the unknown BNP concentration, the spiked BNP concentrations were plotted against relative fluorescence unit (RFU). The unknown concentration of BNP was determined by extrapolating to x-intercept of the resultant linear curve.

### Standard Addition for CNT-TF Sensor

The standard addition method was also used to determine the concentration of BNP in an unknown sample. The sample with unknown BNP concentration (X pg/ml) was spiked with additional BNP to produce X+0, X+1000 and X+2000 pg/ml samples. The three BNP samples and a BNFP sample were added to each of the four chambers of the BSA-blocked test strip. The EIS spectra at 0, 5, 10 and 20 min were collected and fitted using the proposed equivalent circuit in MATLAB. After fitting, the resistances of CNT-TFs in each chamber were collected and the % changes of CNT-TF resistance at 5, 10 and 20 min were calculated relative to the resistance at 0 min. To determine the BNP concentration in the unknown sample, the % change for each chamber was plotted against the spiked BNP concentration. After calibrating the standard plot using BNP-free samples, the unknown BNP concentration was determined by the absolute value of the x-intercept of the extrapolated linear curve. For limit of detection (LOD), 10 BNFP samples were used as blanks. LOD of the CNT-TF sensor was determined using the following equation: LOD=mean+2× standard deviation

### Correlation Between Optical Immunoassay and CNT-TF Sensor

Triplicate samples containing 0, 100, 500, 750, and 1000 pg/mi of BNP diluted in BNFP were tested in both the CNT-TF sensor and optical immunoassay. The calculated BNP concentrations from both methods were plotted against each other to discern correlation.

### Clinical Trial

Twenty-eight plasma samples were collected from 11 patients from Fuwai Hospital in China. The BNP level in each sample was determined using both the CNT-TF sensor and Alere Triage. The correlation between the CNT-TF sensor and Alere Triage was plotted to determine the accuracy of the CNT-TF sensor.

### Detection of NT-proBNP

Antibody immobilization and BSA blocking were performed by following the previously described protocols. A different capture antibody (24E11) that recognizes the amino acids at position 67-76 of NT-proBNP was used. Samples containing 0, 100, 500, 1000, 2000 and 4000 pg/ml NT-proBNP in BNFP were prepared in triplicate. The experimental concentrations of the NT-proBNP samples were determined using the standard addition method mentioned previously. The experimental concentration of NT-proBNP was plotted against its theoretical concentration.

### Results

### Carbon Nanotube Thin Film Characterization

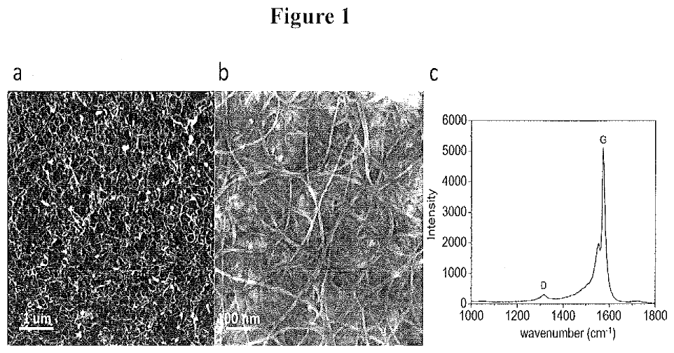

FIGS. 1a and 1b show AFM (atomic force microscopy) and SEM images of the CNT network, respectively. The dimensions of an individual CNT were approximately 1-10 nm in diameter and a few microns in length. The high density of CNT-TF on the silica surface can be observed from the AFM image (FIG. 1a). The Raman spectrum in FIG. 1c shows a 10:1 ratio between G-band from the graphite-like sp² hybridized carbon and D-band from the diamond-like sp³ hybridized carbon. This indicates that the CNT have high purity, high structural integrity and low number of surface defects.

### Device Fabrication and Impedance Measurement

FIG. 2 illustrates step-by-step CNT-TF sensor fabrication. First, the vinyl tape was stacked onto the PET surface and patterned using a vinyl cutter to form a mask for electrodes (FIGS. 2a and 2b). Then 40 nm/200 nm Cr/Au were deposited through an e-beam evaporator and the excess vinyl was peeled off, leaving Au electrodes on the PET surface (FIGS. 2c and 2d). Next, a rectangular vinyl mask was stacked onto the electrodes surface for CNT-TF transfer (FIGS. 2e and 2f). After the vinyl mask was being removed, a well-defined rectangular shape of CNT-TF was formed on the Au electrodes (FIG. 2g). Then a PMMA top cover with four chambers was aligned on the PET surface and the antibodies were immobilized on the CNT-TF surface (FIGS. 2h and 2i). Last, blocking agent was added and the BNP level in the blood plasma sample was tested (FIG. 2j).

The present sensor or point-of-care testing (POCT) device consisted of two parts: a disposable test strip (the biosensor comprising a sensing layer and sample chamber layer) and a miniature electrical readout unit, similar to the commonly used blood glucose meter. The test strip was designed as a two-layer structure as above described. The bottom layer consisted of a fully electric immunosensor array on a low-cost PET substrate. Enlarged metal pads (Au, Ag, AgCl, Pd or any combination thereof) were placed on one end of the strip to connect to the readout electronics. The top layer contained four chambers that guide the blood plasma to the sensor surface. Each immunosensor consisted of a CNT-TF that bridges a pair of gold electrodes. Monoclonal anti-BNP antibodies (50E1), which recognize and bind to amino acids 26-32 of the BNP molecule, were immobilized on the CNT network. The dissociation constant of the 50E1 antibody was calculated as 3.59 nM. The BNP-antibody binding events on the CNT surface modulate CNT conductivity and, thus, induce a linearly proportional change in overall electrical conductance. Conductivity may be modulated as a result of deformation of the antibody after BNP binding which alters the doping effect (i.e. charge transfer between the CNT and antibody), or as a result of electrostatic gating due to the charge redistribution on the CNT surface (e.g. positive charge density).

The in-house, custom-made reader unit was based on AC potential and detects current within the following parameters: (1) the CNT resistance ranged from 2 to 10 kΩ, (2) the frequency ranged from 80-60,000 Hz with increments of 100 Hz, and (3) the size of the reader was 4×6 inch. The CNT/immunoassay strip plugs into the reader unit. The reader collected the data and transferred it to the computer screen before conducting data analysis.

### Characterization of CNT-TF Sensor

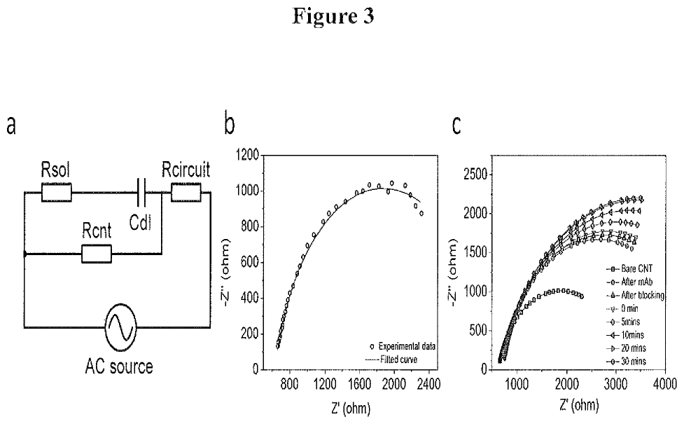

FIG. 3a shows a simplified equivalent circuit for the CNT-TF sensor neglecting the contribution of Warburg impedance. The equivalent circuit included CNT-TF resistance (Rₑₙₜ), plasma solution resistance (Rₛₒₗ), gold-electrode resistance (R_{circuit}), and double layer capacitor (C_{dl}). The Rₑₙₜ was in parallel with Rₛₒₗ & C_{dl} because CNT-TF provided an alternative pathway for the electron flow. Based on the equivalent circuit, the tested EIS spectrum was fitted using MATLAB code (FIG. 3b). The imaginary part, the real part and the impedance were fitted together in the MATLAB code using Isqnonlin curve fitting. The perfectly fitted Nyquist plot in FIG. 3b suggests that the proposed equivalent circuit sufficiently imitates the electrochemistry in the CNT-TF sensor. FIG. 3c illustrates the Nyquist plot of the CNT-TF sensor during sensor preparation, 50E1 antibody immobilization, 5% BSA blocking, and BNP sample testing. The increasing diameter of the semi-circle at each step indicates that the resistance of the CNT-TF increased as more molecules adsorbed onto the CNT-TF surface. The CNT-TF resistance values and its percentage changed at each step of the sensor preparation and sample testing. The CNT-TF sensor reached saturation after 20 minutes of sample incubation, demonstrating that the sensor was ready for detection. The 20-minute detection time was also within the BNP's half-life of 23 minutes, showing the sensor's superior response time. The summarized CNT-TF resistance (Rₑₙₜ) percentage change at 5, 10 and 20 minutes after BNP addition was used as the detection signal.

### Optical Immunoassay Vs CNT-TF Sensor

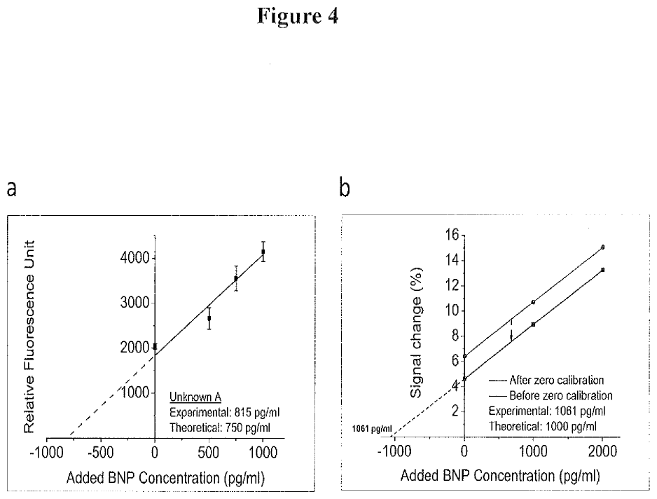

Standard addition is a very useful method for detecting analyte of unknown concentration in a complex matrix such as the human plasma. The standard addition method was first validated through optical immunoassay, which is considered the gold standard for BNP detection. As shown in FIG. 4a, an unknown sample was experimentally determined using an optical immunoassay to include 815 pg/mL BNP, which had an 8.67% error from the theoretical BNP concentration of 750 pg/mL. Thus, it was concluded that the standard addition method can accurately determine unknown concentrations of BNP while eliminating background signal from the plasma matrix. In this study, known quantities of BNP were added onto hydrophobic PMMS and dried before adding the unknown sample. Hence, the BNP successfully re-dissolved upon contact with plasma.

FIG. 4b illustrates how the standard addition analysis was able to accurately determine unknown BNP concentrations on the CNT-TF sensor. BNFP was added to one of the four chambers to remove the background signal derived from plasma. The other three chambers with unknown (X) and spiked BNP samples (X+1000 and X+2000 pg/mL BNP) showed a linear relationship with the concentration of BNP. After subtracting background signal from BNFP, a self-calibration curve was prepared and the unknown BNP concentration (X) was determined by extrapolating the curve to the x-intercept. In this example, the BNP concentration was determined to be 1061 pg/mL, which had a 6% error from the theoretical value of 1000 pg/mL. Thus, the standard addition method was again proven to be advantageous for the CNT-TF sensor. It does not only act as a calibration curve to determine unknown concentrations of BNP, but also permits elimination of background signal from the complex plasma matrix.

To determine the LOD of the CNT-TF sensor, 10 BNFP samples without BNP were tested as blanks. The mean and standard deviation of the 10 tests were 3.24 pg/mL and 6.44 pg/mL, respectively. The LOD was then determined as 16 pg/mL, comparable to other commercial BNP-monitoring techniques such as Alere Triage Pro™ and Abbott i-Stat™.

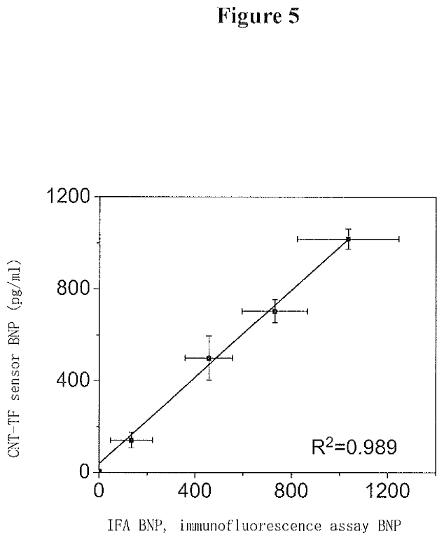

Next, the accuracy and precision of the CNT-TF sensor was examined by comparing it to an optical immunoassay. The tested BNP concentrations in blood plasma were 0, 100, 500, 750, 1000 pg/mL, within the clinically relevant range for HF patients. Each prepared BNP plasma sample was divided into two and simultaneously tested in the two systems. FIG. 5 shows the linear correlation between the two methods. The coefficient of determination (R² 0.989) indicated high accuracy and sensitivity even to low concentrations of BNP (100 pg/mL). The coefficient of variation (CV) values of both methods was less than 20%.

### Clinical Trial

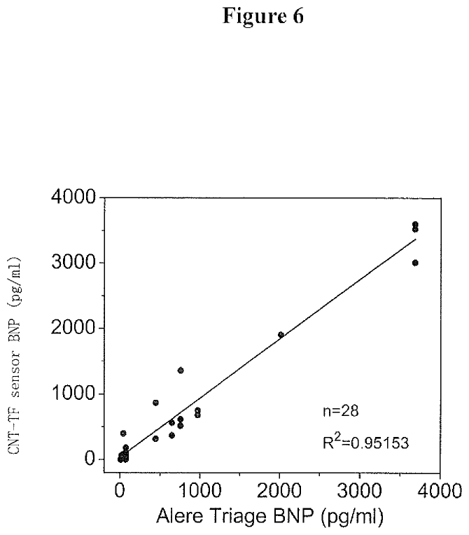

The CNT-TF sensor was further tested in clinical trials with on-site patient plasma. For reference purposes, the CNT-TF device was compared to the Alere Triage® system, a well-established commercial product for monitoring BNP in central labs. Twenty-eight venous blood samples were drawn from 11 volunteers from Fuwai hospital in China and centrifuged to extract the blood plasma. Each plasma sample was divided into two: one for the CNT-TF sensor and the other for the Alere Triage® system. The tested BNP concentration ranged from 10-4000 pg/mL in patient plasma. FIG. 6 shows the correlation between the BNP concentrations measured by both CNT-TF sensor and the Alere Triage® system. The linear regression between the CNT-TF sensor and the Alere Triage® system (0.95153; p<0.0001) indicated comparable performance. Compared to the Alere Triage® system and other existing optical BNP sandwich (capture and detection antibodies) immunoassays, the CNT-TF sensor uses a single antibody for BNP capture, has high intrinsic sensitivity and direct response to BNP-antibody binding. These characteristics give the CNT-TF sensor the advantage of detecting BNP without the need for any additional labeling or amplification molecules such as fluorophores or enzymes. Furthermore, the signal detection arises only upon BNP-binding events, and no washes are needed. Overall, the CNT-TF sensor was shown to exhibit exceptional sensitivity when standard addition is implemented, compared to other optical methods that require washing to eliminate false-positive signals.

### Detection of NT-proBNP

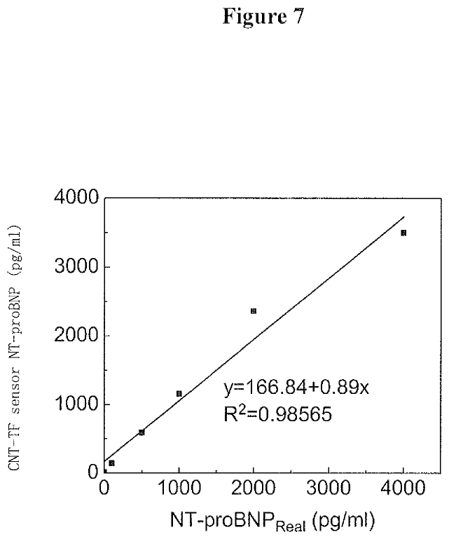

Finally, the CNT-TF sensor was used to detect NT-proBNP, which is one of the least concentrated HF biomarkers with the shortest half-life. NT-proBNP antibodies (24E11 antibodies) were attached to the CNT-TF device as described above. These antibodies detect the amino acids at position 67-76 of NT-proBNP. Standards of NT-proBNP at 4000, 2000, 1000, 500 and 100 pg/mL in BNFP were used to compare theoretical and empirical values. FIG. 7 illustrates that the device is highly accurate with a linear regression slope of 0.89. The ability of the CNT-TF sensor to accurately detect NT-proBNP confirms that the device has utility for the detection of other biomarkers, including detection of other HF biomarkers, as well as biomarkers for other diseases and microbial infections.

### Example 2—Evaluation of the Precision of the Biosensor System

Protocol: The precision of the biosensor system was evaluated over at least 20 days (not necessarily consecutive working days), two runs per testing day, with a minimum of two hours separation between run and two replicates per sample per run. Based on this design, a total of 80 measurements were obtained for each sample (20 or more days×2 runs per day×2 results per run). Throughout the test, a single instrument at a single site was used.

On the morning of day 1, the first run was performed using a fresh prepared commercial BNP-free plasma sample spiked with 500 pg/ml BNP. In the afternoon of day 1, the second run was performed using another fresh prepared commercial BNP free plasma spiked with 500 pg/ml BNP. Replicates were measured in each run. This was repeated on another 19 days. The EIS data for each replicate was recorded in a nested data record table.

ANOVA and imprecision analysis was performed on the final results to evaluate the precision of the system. Table 1 shows the Two-Way nested ANOVA of the 20 days×2 runs×2 replicates study. By dividing the mean square for day by the mean square for Run (Day), an F value of 1.24 was obtained, which was less than the P value of 2.12 for 19 and 20 degrees of freedom at the 0.05 significance level. By dividing the mean square for Run (Day) by the mean square for Error, an F value of 0.84 was obtained, which is less than the critical value of 1.84 for 20 and 40 degrees of freedom at 0.05 significance level. In conclusion, the within-day and within-run variation were not significant enough to affect the precision of the system.

TABLE 1
Two-Way nested ANOVA of the 20 days × 2 runs × 2 replicates study

| Source of | | | | | |
|---|---|---|---|---|---|
| Variation | SS | df | MS | F | P-value |
| Day | 125068.95 | 19.00 | 6582.58 | 1.24 | 2.12 |
| Run (Day) | 106229.97 | 20.00 | 5311.50 | 0.84 | 1.84 |
| Error | 252525.99 | 40.00 | 6313.15 | | |
| Total | 483824.90 | 79.00 | | | Alpha = 0.05 |

The accuracy of the biosensor is summarized in Table 2 which shows detection of a mean value of 502.8 pg/ml of the BNP analyte and a % CV of 15.56%.

TABLE 2
Total with-in laboratory imprecision

| Sample Description | N | Mean Value | SD | CV |
|---|---|---|---|---|
| BNP Spiked Plasma | 80 | 502.8 | 78.26 | 15.56% |

Relevant portions of references referred to herein are incorporated by reference.

1. A biosensor useful for the detection of a target analyte in a fluid biological sample comprising:
i) a first sensing layer comprising at least a pair of metal electrodes on an inert substrate, wherein the electrodes are linked by an electrically conductive bridge having immobilized on the surface thereof analyte-reactive compounds, wherein the electrically conductive bridge comprises a material that responds to binding of target analyte to the analyte-reactive compound with a change in an electrochemical or electrical signal at the electrically conductive bridge; and, optionally,
ii) a second layer comprising one or more sample chambers for placement on the first layer, wherein the sample chambers are adapted to receive a fluid sample and direct the sample to the electrically conductive bridge.

2. The biosensor of claim 1, wherein the substrate comprises a thermoplastic polymeric substrate and the electrodes comprise carbon, gold, silver, platinum or a combination thereof.

3. The biosensor of claim 1, wherein the electrically conductive bridge comprises a carbon-based material, silica or molybdenum disulfide (MoS₂).

4. The biosensor of claim 1, wherein the electrically conductive bridge comprises carbon nanotubes.

5. The biosensor of claim 1, wherein the analyte-reactive compound is selected from the group consisting of an oligonucleotide, a monoclonal or polyclonal antibody, a protein or peptide ligand, a receptor, an enzyme or a substrate.

6. The biosensor of claim 1, wherein the analyte-reactive compound is immobilized on the electrically conductive bridge by covalent linkage, electrostatic attraction, ionic bonding, hydrogen bonding, or van der Waals' forces.

7. The biosensor of claim 1, wherein the biological sample is a bodily fluid selected from blood, plasma, serum, saliva, urine, tears, breast milk, cerebrospinal fluid, amniotic fluid and ascitic fluid.

8. The biosensor of claim 1, comprising means to electrically connect the first sensing layer to a detection device.

9. The biosensor of claim 1, comprising an array of electrodes.

10. The biosensor of claim 1, comprising multiple working electrodes connected to a common counter electrode.

11. The biosensor of claim 1, wherein the second chamber layer comprises a single inlet that branches into 2 or more sample chambers that deliver sample to the first sensing layer for contact with the electrically conductive bridge(s) joining the electrodes.

12. The biosensor of claim 1, wherein the substrate comprises an upper face and a lower face, and electrodes are on both the upper and lower face.

13. The biosensor of claim 1, which is encapsulated in a protective casing that compresses the first and second layers of the biosensor together.

14. A point of care device useful for the detection of a target analyte in a fluid biological sample comprising:
i) a biosensor having a first sensing layer comprising at least a pair of metal electrodes on an inert substrate, wherein the electrodes are linked by an electrically conductive bridge having immobilized on the surface thereof analyte-reactive compounds, wherein the bridge comprises a material that responds to target analyte binding with a change in an electrochemical or electrical signal at the electrically conductive bridge; and, optionally, a second layer comprising one or more sample chambers for placement on the first layer, wherein the sample chambers are adapted to receive a fluid sample and direct the sample to the electrically conductive bridge; and
ii) a detection device that forms an electrical circuit with the biosensor, provides an electrical signal over a range of frequencies and is adapted to detect an electrochemical or electrical change at the electrically conductive bridge in the presence of target analyte using electrochemical impedance spectroscopy on application of the electrical signal.

15. The device of claim 14, wherein the detection device applies an electrical signal having a potential of less than 50 mV over a range of frequencies of 80-60,000 Hz.

16. The device of claim 14, wherein the detection device applies an electrical signal having a potential of 2-10 mV over a range of frequencies of 200 to 10,000 Hz.

17. The device of claim 14, wherein the electrically conductive bridge comprises a carbon-based material, silica or molybdenum disulfide (MoS₂).

18. The device of claim 17, wherein the electrically conductive bridge comprises carbon nanotubes.

19. The device of claim 14, wherein the detection device provides an output indicating the presence or absence of target analyte in the sample.

20. A method of detecting a target analyte in a patient sample comprising:
i) applying the sample to the biosensor of claim 1;
ii) collecting electrochemical signal data from the biosensor in the presence of the sample over a range of frequencies; and
iii) analyzing conductivity, resistance and/or capacitance based on the electrochemical signal data using electrochemical impedance spectroscopy (EIS) and determining the presence of target analyte in the sample when the impedance or overall resistance is different in the absence and presence of the sample.
